(19) United States
(12) Patent Application Publication
(10) Pub. No.: US 2003/0143202 A1
(43) Pub. Date: Jul. 31, 2003

(54) Anemia

(76) Inventors: Katie (Mary) Binley, Oxford (GB); Susan Mary Kingsman, Oxford (GB); Stuart Naylor, Oxford (GB)

Correspondence Address: FROMMER LAWRENCE & HAUG, 745 FIFTH AVENUE- 10TH FL., NEW YORK, NY, 10151, US

(21) Appl. No.: 10/066,218

(22) Filed: Feb. 1, 2002

(30) Foreign Application Priority Data
Jan. 31, 2002 (GB) 0202252.3

(51) Int. Cl. A61K 48/00; C12N 15/86

(52) U.S. Cl. 424/93.2; 435/456; 514/44

(57) ABSTRACT

Disclosed is a vector containing a nucleic acid sequence encoding erythropoietin (Epo) in operable linkage with an HRE expression control sequence, as well as uses thereof; for instance, in preparing a medicament, as well as in methods for treating anemia in a patient in need thereof. The method can involve administering to the patient a vector comprising a nucleic acid sequence encoding erythropoietin (Epo) in operable linkage with an HRE expression control sequence, wherein expression of Epo is physiologically regulated such that hematocrit levels of the patient are corrected and maintained.

### REFERENCE TO RELATED APPLICATIONS AND INCORPORATION BY REFERENCE

This application claims priority from British Application No.______, filed Jan. 31, 2002 (UK Attorney Docket No. P013473GB).

Reference is made to: U.S. Pat. No. 6,265,390 (Methods For Expressing Nucleic Acid Sequences Using Nucleic Acid Constructs Comprising Hypoxia Response Elements), filed Feb. 22, 1999, U.S. Pat. No. 5,942,434 (Nucleic Acid Constructs Comprising Hypoxia Response Elements), filed Dec. 12, 1996, PCT/GB95/00322, filed Feb. 15, 1995 and published Aug. 17, 1995 as WO 95/21927 (Targeting Gene Therapy), GB application serial no. 9402857, filed Feb. 15, 1994, and WO 00/17371, filed Sep. 22, 1999; nationally processed in the U.S. as U.S. Ser. No. 09/787,562.

Each of the foregoing applications and patents and articles, and each document cited or referenced in each of the foregoing applications and patents and articles, including during the prosecution of each of the foregoing applications and patents (“application and article cited documents”), and any manufacturer's instructions or catalogues for any products cited or mentioned in each of the foregoing applications and patents and articles and in any of the application and article cited documents, are hereby incorporated herein by reference. Furthermore, all documents cited in this text, and all documents cited or referenced in documents cited in this text, and any manufacturer's instructions or catalogues for any products cited or mentioned in this text or in any document hereby incorporated into this text, are hereby incorporated herein by reference. Documents incorporated by reference into this text or any teachings therein may be used in the practice of this invention. Documents incorporated by reference into this text are not admitted to be prior art. Furthermore, authors or inventors on documents incorporated by reference into this text are not to be considered to be “another” or “others” as to the present inventive entity and vice versa, especially where one or more authors or inventors on documents incorporated by reference into this text are an inventor or inventors named in the present inventive entity.

### FIELD OF THE INVENTION

The present invention relates to an improved vector system and the use of said vector in the treatment of chronic anemia. In particular, the present invention relates to the construction and use of a novel vector system which directs regulated erythropoietin (Epo) gene therapy in a manner which physiologically corrects the hematocrit levels in a patient in need of such treatment.

### BACKGROUND

Tissue hypoxia is the key physiological signal for increasing erythropoiesis via a direct effect on the expression of the Epo gene (Maxwell et al. (1993) Kidney Int. 44: 1149-1462). Upon hypoxic exposure, the kidney, and to a lesser extent, the liver increase Epo synthesis up to 1000-fold. Epo then circulates through the blood to the bone marrow where it promotes maturation of erythrocytes (Ebert et al. (1999) Blood 94: 1864-1877). Defining the mechanism of hypoxic induction of Epo production led to the identification of a potent regulatory sequence in the Epo enhancer that bound a transcription factor. The factor was identified as a heterodimer with independently regulated subunits termed hypoxia inducible factor-1 (HIF-1). HIF-1 is ubiquitously expressed and the consensus HIF-1 binding sequences exist in a number of genes in addition to Epo and are termed hypoxia responsive enhancers or elements (HRE) (Wenger et al. (1997) Biol. Chem. 378: 609-616). Defining the hypoxic regulation of Epo has led to an advancement in the general understanding of the cellular response to hypoxia. In fact, various natural and synthetic HRE containing promoters have been used to direct heterologous gene expression in response to hypoxia, for example in tumour cells, muscle and macrophages (U.S. Pat. Nos. 6,265,390 and 5,942,434, Binley et al. (1999) Gene Ther. 6: 1721-1727, Griffiths et al. (2000) Gene Ther. 7: 255-262, Shibata et al. (2000) Gene Ther. 7: 493-498).

Chronic anemia occurs when there is a decrease in oxygen carrying capacity of the blood due to a shortage of red blood cells (RBC). One of the underlying causes of chronic anemia is a failure in the production of the protein hormone Epo that regulates the formation of RBCs. This results in a dramatic reduction in the number of circulating RBCs, measured by the hematocrit. This is particularly evident in end stage renal disease (ESRD), cancer and some chronic inflammatory diseases such as rheumatoid arthritis (Goodnough et al. (2000) Blood 96: 823-833, Bron et al. (2001) Semin. Oncol. 28: 1-6). The reduction in RBCs reduces the ability of the blood to oxygenate tissues causing tissue hypoxia. The pathophysiological responses correlate with the severity of the hypoxia and range from fatigue and hypertension through to cardiovascular disease and heart failure. Current treatment of this class of anemia includes the regular intravenous administration of recombinant human Epo (rhEpo) several times a week. However, on a cost and convenience basis this treatment regime may not be suitable for all indications particularly in severe chronic anemia that requires continuous and frequent treatment. Consequently, there has been considerable interest in developing a gene therapy strategy for the delivery of Epo whereby the single administration of the Epo gene would ensure the long-term delivery of Epo.

To this end, numerous methods for Epo gene therapy were investigated as a means to find alternatives to rhEpo protein therapy. These methods utilized a range of gene therapy delivery vehicles such as plasmid DNA, and viral vectors (U.S. Pat. No. 6,211,163, Osada et al. (1999) Kidney International 55: 1234-1240, Dalle et al. (1997) Hematol. Cell Ther. 39: 109-113, Bohl et al. (1998) Blood 92: 1512-1517, EP 1013288, Rudich et al. (May 2000) J. Surg. Res. 90: 102-108, Zhou et al. (May 1998) Gene Ther. 5: 665-670, Svennson et al. (October 1997) Hum. Gene Ther. 8: 1797-1806, Beall et al. (March 2000) Gene Ther. 7: 534-539, Payen et al. (March 2001) Exp. Hematol. 29: 295-300, Tripathy et al. (November 1994) PNAS 91: 11557-11561, Klinman et al. (March 1999) Hum. Gene Ther. 10: 659-665, Maione et al. (April 2000) Hum. Gene Ther. 11: 859-868, Descamps et al. (August 1994) Hum. Gene Ther. 5: 979-985, Maruyama et al. (March 2001) Gene Ther. 8: 461-468, Verma (1999) J. Gene Med. 1: 64-66, Kessler et al. (November 1996) PNAS 93: 14082-14087, Seppen et al. (August 2001) Blood 98: 594-596), or transfer of ex vivo modified Epo expressing cells (Bohl et al. (1997) Nat. Med. 3: 299-305, Osborne et al. (August 1995) PNAS 92: 8055-8058, Villeval et al. (August 1994) Blood 84: 928-933, Serguera et al. (1999) Hum. Gene Ther. 10: 375-383).

However, these methods failed to demonstrate any genuine therapeutic effect on chronic anemia. This is because the Epo gene has been delivered to either normal animals (Rudich, Beall, Serguera, and Bohl (1998), as above), or to inappropriate models such as beta-thalassemic mice (Villeval (1994), Payen (2001), as above, Bohl et al. (2000) Blood 95: 2793-2798, Dalle et al. (1999) Gene Ther. 6: 157-161), or to acutely anemic animals, for example where the kidneys have been severely damaged (Hamamori et al. (1995) J. Clin. Invest. 95: 1808-1813). As such, measurements of the hematocrit in these models are not a true indicator of therapy in that they are taken against baseline normal hematocrit levels or as a transient rise in the acute anemia environment. Furthermore, in many of these models, the introduction of the Epo gene results in a relentless rise in the hematocrit causing the opposite of anemia, polycythemia, a state characterized by having too many RBCs (Bohl et al. (2000), as above), which often requires frequent phlebotomy to reduce the risk of thrombosis (Rudich (2000), Zhou (1998), as above). It is believed that a consistently high hematocrit increases the risk of hypertension, heart failure and thrombosis. Thus, the state of the art represents that a method for providing meaningful Epo gene therapy in a clinical respect is both necessary and desirable.

In attempts to meet the need for regulating Epo gene therapy, researchers have developed systems which can be switched off by using a regulated promoter such as the Tetracycline or Rapamycin responsive promoters. However, to date, this approach has only been demonstrated to regulate the hematocrit above the normal baseline rather than to maintain normal levels (Ye et al. (1999) Science 283: 88-91, Bohl (1998), Rendahl (1998), and Bohl (1997), as above). In addition, the use of these extrinsic regulation systems in a clinical setting would require long-term maintenance and control of Epo gene expression, both of which would be costly and cumbersome, particularly since the addition of the pharmacological regulatory agents may interfere with other patient medications.

Setoguchi et al. (Blood, 94: 2946-2953, Nov. 1, 1994) utilize an adenoviral construct with human Epo gene (the gene itself including its 3′ 150 bp enhancer). The organization of the construct exploits the enhancer at the 3′ end of the human Epo gene in its natural position, the gene of which is under control of the adenoviral MLP promoter. The disadvantage with this approach is that it fails to produce physiologically-regulated expression of Epo.

Aebischer et al. (U.S. Pat. No. 5,952,226, and Human Gene Therapy, 8(16): 1840-1841, Nov. 1, 1997) utilize an encapsulated cellular implant to express the Epo gene.

Accordingly, there remains a need in the art for a vector system suitable for the regulation of Epo which when functioning reproduces the physiological regulation of Epo, and thus allows patient hematocrit levels to be therapeutically corrected and maintained.

### SUMMARY OF THE INVENTION

The present invention provides an improved vector system suitable for the therapy of chronic anemia.

Thus in a first aspect, the present invention provides a vector system for the physiological regulation of Epo, the vector system comprising a nucleic acid sequence encoding erythropoietin (Epo) in operable linkage with an HRE expression control sequence, wherein the HRE expression control sequence includes two or more HRE expression control sequences, and the vector system, when administered to a host provides for the physiological regulation of Epo.

In a further aspect, the present invention provides the use of a vector system comprising a nucleic acid sequence encoding erythropoietin (Epo) in operable linkage with an HRE expression control sequence in the preparation of a medicament for the prophylaxis and/or treatment of anemia wherein the expression of Epo is physiologically regulated.

Organization of the construct of the present invention positions an HRE at the 5′ end of the construct in operable linkage with the promoter such that the HRE & promoter (creating a hypoxia inducible promoter/expression control sequence) controls expression of the Epo gene as set forth in FIG. 1A of this specification. In contrast to the present invention, the organization of the construct of Setoguchi et al. (Blood, 94: 2946-2953, Nov. 1, 1994) exploits the enhancer at the 3′ end of the human Epo gene in its natural position, the gene of which is under control of the adenoviral MLP promoter. Furthermore, the use of the construct as reported in Setoguchi et al., fails to segue to the surprisingly enhanced effects of the present invention reported herein, i.e., the near-perfect physiologically-regulated expression of Epo in the anemic environment of an art-recognized animal model.

Aebischer et al. (U.S. Pat. No. 5,952,226, and Human Gene Therapy, 8(16): 1840-1841, Nov. 1, 1997) utilize an encapsulated cellular implant to express the Epo gene. In contrast to the present invention, Aebischer et al. set forth an ex vivo approach rather than an in vivo approach, and furthermore, fail to teach or suggest the surprisingly enhanced effects of the present invention reported herein, i.e., the near-perfect physiologically-regulated expression of Epo in the anemic environment of an art-recognized animal model. Disadvantageously, the encapsulated cell technique of Aebischer et al. involves the surgical implant and explant of the capsule, whereas, in vivo administration of a gene therapy vector, as in the present invention, overcomes the need to surgically implant or explant the vehicle delivering the therapeutic gene.

In a further aspect still, the present invention provides the use of a vector comprising a nucleic acid sequence encoding erythropoietin (Epo) in operable linkage with an HRE expression control sequence in the preparation of a medicament for maintaining and correcting the hematocrit levels of a patient.

According to the above aspects of the invention, the HRE expression control sequence is advantageously associated with a promoter, preferably an HRE promoter within a vector system, to create a HRE promoter/expression control sequence. At least one HRE and/or HRE promoter/expression control sequence is in operable linkage with an Epo coding sequence. A vector system according to the invention directs the regulation of Epo expression in a surprising and unexpected manner and reproduces the near perfect physiologically-regulated expression of Epo in the anemic environment of an art recognized animal model.

Of course, the inventive vector is also useful for in vitro Epo expression, e.g., by contacting the vector with a suitable cell under conditions which allow for expression of the Epo, and optionally harvesting the expressed Epo, which can be used in the same fashion as other protein Epos.

Advantageously, the vector system can be any vector system, such as a viral vector system, e.g., retroviral, lentiviral, adenoviral, adeno-associated viral, and the like, or a non-viral vector system such as naked DNA, lipid complexed-DNA, or biolistic DNA delivery, DNA plasmid, and the like.

Advantagously, the vector system can be administered by any known route of delivery, such as intramuscular, intravascular, subcutaneous, or intraperitoneal administration. The skilled artisan, based on this disclosure and the knowledge in the art, including documents cited herein, can determine a route of administration, without any undue experimentation, including by considering such factors as the particular species of the patient and the particular vector.

Advantageously, the HRE can further be in operable linkage with any promoter, such as a viral promoter, or cellular promoter, that can be constitutive, inducible, or tissue-specific in function.

Advantageously, the Epo nucleic acid sequence can be synthetic or can be derived from any species of Epo, such as human Epo, non-human primate Epo, canine Epo, feline Epo, porcine Epo, bovine Epo, equine Epo, ovine Epo, and murine Epo.

Advantageously, the patient to be treated for chronic anemia may be any patient of a species such as human, non-human primate, canine, feline, porcine, bovine, equine, ovine, and murine. Advantageously, the present invention finds use in a clinical setting, which can include use in the veterinary field providing treatment to companion animals as well as farm animals. Advantageously, the Epo coding sequence and patient to be treated can be of the same species or of a different species.

The terms “comprises”, “comprising”, and the are open, inclusive terms which do not exclude further elements; they can thus mean “includes”, “including” and the like.

These and other embodiments are disclosed or are obvious from and encompassed by, the following Detailed Description.

### BRIEF DESCRIPTION OF THE FIGURES

The following Detailed Description, given by way of example, but not intended to limit the invention to specific embodiments described, may be understood in conjunction with the accompanying Figures, incorporated herein by reference, in which:

FIG. 1 (FIGS. 1A-C) shows: (A) Diagrammatic representation of recombinant AAV-2 vectors used for this study. The AAV-CMVEpo and AAV-HREEpo virus vectors only differ in the nature of the promoter sequence. ITR indicates AAV-2 inverted terminal repeats; CMV, immediate/early promoter enhancer elements from CMV; HRE, hypoxia responsive promoter mEpo, murine erythropoietin; SV40 (pA); polyadenylation signal from the SV40 virus; Stuffer DNA, fragment of the 3′ β-galactosidase gene to ensure genome size is over 4 kb. (B) Proliferation of splenocytes incubated with supernatants from HT1080 cells transfected with pCMV/HRE-Epo plasmids. The assay shows the increased proliferation of the splenocyte cells when exposed to the supernatant from the pHRE-Epo transfected cells that have been exposed to hypoxia. The negative control consists of untreated cells, rhEpo indicates recombinant human Epo used as a positive control. Data are the mean relative light units per second values +/−SD of 3 samples. (C) Hypoxia regulated Epo expression is maintained in a rAAV vector. T47D cells were transduced with rAAV-2 vectors, AAV-CMVEpo and AAV-HREEpo. Supernatants were harvested 1 day (grey bars) and 4 days (white bars) post hypoxic treatment and analysed in an Epo ELISA assay. Data are the mean mIU/ml epo values +/−SD of 3 samples. The dotted line represents the detectable threshold of the assay.

FIG. 2 (FIGS. 2A-D) shows: The skeletal muscle in the Epo-Tagh transgenic mice shows increased vascularity compared to the parental mice. The skeletal muscle from Epo-TAgh transgenic (B and D) and parental wild type (A and C) mice were sectioned transversely and immunologically stained for the endothelial cell marker, CD31 (A and B) and the angiogenic factor, VEGF165 (C and D).

FIG. 3 (FIGS. 3A-B) shows: (A) AAV-HREEPO treated EPO-TAg^{h} transgenic mice display physiological correction of the haemtocrit. Closed symbols are EpoTAg^{h} groups and open symbols are wild type groups (Closed circle) EpoTAg^{h} group. (Open circle) wild-type group. (Closed square) EpoTAg^{h} treated with AAV-CMVGFP. (Open square) wild-type treated with AAV-CMVEpo. (Closed diamond) EpoTAg^{h} treated AAV-CMVEpo. (Open diamond) wild-type treated with AAV-CMVEpo. (Closed triangle) EpoTAg^{h} treated with AAV-HREEpo. (Open triangle) wild-type treated with AAV-HREEpo. Haematocrits are plotted as a mean value for 6 animals in each treatment group +/−SD. (B) Expansion of the haematocrit data from the mice treated with the AAV-HREEpo data. (Open square) EpoTAg^{h} mice (Open triangle) wild-type mice. Haematocrit data from each individual animal treated with the AAV-HREEpo vector is plotted.

FIG. 4 (FIGS. 4A-B) shows: (A) Analysis of the heart and spleens in the EpoTAg^{h} and wild-type mice before and after treatment with rAAVEpo vectors. (White bar) EpoTAg^{h} mice (Black bar) wild-type mice (Pale grey bar) EpoTAg^{h} mice treated with AAV-CMVEpo (Dark grey bar) wild-type mice treated with AAV-CMVEpo. The average weights of organs is plotted +/−the standard deviation (n=3). (B) Histological analysis of the heart in untreated and rAAV-Epo treated Epo-Tag and wild-type mice. A. Epo-TAg^{h} heart showing enlarged LV. B. wild type heart. C. Epo-TAg heart at day 70 post AAV-CMVEpo treatment. D. Wild-type heart at day 70 post AAV-CMVEpo treatment.

FIG. 5 (FIGS. 5A-5D) shows: EM pictures of the hearts showing partial reversal of the cardiac hypertrophy.

### DETAILED DESCRIPTION

Correcting anemia is a clinically important challenge as chronic anemia can lead to congestive heart failure that can be fatal if left untreated. The present invention achieves physiologically-regulated expression of the Epo gene and correction and maintenance of the hematocrit in a clinically relevant anemic environment. The present invention provides an optimized vector system comprising an HRE expression control sequence and optionally an HRE promoter in operable linkage with an Epo coding sequence, which vector system directs regulated Epo gene therapy in a surprising and unexpected manner by physiologically correcting and maintaining the hematocrit in a patient in need thereof.

The invention is also directed to the use of a vector system as herein described in the preparation of a medicament for the prophylaxis and/or treatment of chronic anemia, more specifically by mimicking the physiologically regulated expression of Epo.

The present invention is further directed to the use of a vector system as herein described in the preparation of a medicament for correcting and maintaining hematocrit levels in a patient.

With regard to the physiological correction and maintenance of the hematocrit level, it is meant for maintenance to encompass art-recognized treatment guidelines for chronic renal failure which seek to maintain hematocrit levels at 30-33% of the normal range of the hematocrit. (Kaufman et al. (1998) N Engl J Med 339: 578-583.) Normal levels of the hematocrit for males are 39-52%, and for females are 35-47%. (Anemia Work Group. (1997) NKF-DOQI clinical practice guidelines for the treatment of anemia of chronic renal failure. Am J Kidney Dis. 30(4 suppl 3): S 192-S240.) Although, such guidelines recognized there was an increase in mortality if patients were maintained in the normal range, this was thought to be due to the effects of poor dosing control of Epo when provided by the current i.v./s.c. injection regimen. The guidelines also recognized that it can take some weeks for the effects of adjustments to appear making dose adjustment extremely difficult. (Asha et al. (1993) Am J Kid Dis. 22(2 suppl 1): 23-31.) Further, the rapid rise in hematocrit was thought to be causally related to the hypertension seen in a large proportion of patients treated. Raine et al. (1991) Am J Kidney Dis. 18(4 suppl 1): S76-S83, Besarab et al. (1998) N Engl J Med. 339: 584-590, Watson et al. (1990) Am J Med. 89: 432-435.)

Thus, the present invention provides advantages over the aforementioned treatment paradigms, in that the method for Epo gene therapy of chronic anemia by administration of a vector system comprising an HRE expression control sequence in operable linkage with a gene encoding Epo, does not lead to rapid fluctuations, rather it provides a smoother restoration of the hematocrit described by the slow rise and smooth plateau of the hematocrit. This plateau of the hematocrit can be in the normal range of the hematocrit or it may be in the therapeutic range recognized by the aforementioned treatment guidelines. This slow rise and smooth plateau of the hematocrit is not possible with any other Epo therapies. In effect, the present invention offers a better clinical outcome than is possible with other Epo therapies known in the art.

The present invention provides for the use of HREs, or hypoxically-inducible promoters/enhancers (expression control sequences), such as HREs derived from Epo, PGK-1 (EMBL database, accession no. M18735, at nucleotides 631 to 654 and 634 to 651), and LDH-A genes. The HREs of the invention may be chosen from those referred to herein, or they may be other HREs. It is expected that other hypoxically-inducible promoters or enhancers will be discovered as it has been shown that oxygen-sensing systems are widespread in mammalian cells and many genes are likely to be under hypoxic control (U.S. Pat. No. 6,265,390).

Advantagously, the nucleic acid construct according to the invention comprises at least one HRE which confers hypoxic inducibility on the expression control sequence. There may be, for example, two or more HREs linked so as to increase hypoxic inducibility, and thus to increase the induction of the gene or genes under hypoxia. HREs may be chosen from among those referred to herein, or they may be other HREs. Oxygen-sensing systems are widespread in mammalian cells, and it is expected that other HREs having the fundamentally conserved structure and hypoxic inducible function, will be discovered (U.S. Pat. No. 6,265,390).

The construct according to the invention may comprise more than one, e.g., three or more copies of one of the Epo, PGK, LDH-A, or other HRE sequence given above. Additionally or alternatively, a longer portion of the Epo, PGK-1, LDH-A, or other enhancer or flanking sequence may be used in the construct, which longer portion comprises the HRE and part of the surrounding sequence (U.S. Pat. No. 6,265,390, as above). It is noted that regions of the Epo enhancer sequence have been well characterized (mouse Epo enhancer: EMBL accession no. X73471, Maxwell et al. (1993), U.S. Pat. No. 6,265,390, as above, and Semenza et al. (1992) PNAS 88: 5680-5684, and Blanchard et al. (1992) Mol. Cell. Biol. 12: 5373-5385).

The present invention provides for HREs that may be chosen so as to be operative in particular tissues or cell types to be targeted therapeutically, or they may be chosen to work in a wide range of tissues or cell types. Advantageously, the HRE of the present invention can be further in operable linkage with a promoter, such as a viral or cellular promoter. The HRE of the invention finds use with constitutive promoters such as cytomegalovirus (CMV) promoter, SV40 early promoter, mouse mammary tumor virus LTR promoter, adenovirus major late promoter (MLP), and rous sarcoma virus (RSV) promoter, inducible promoters such as murine metallothionein promoter, and tissue-specific promoters. Such promoter sequences are commercially available from, e.g., Stratagene (San Diego, Calif.). As to cytomegalovirus promoters, mention is made of U.S. Pat. Nos. 6,156,567 and 6,090,393, involving truncated CMV promoters, as well as U.S. Pat. Nos. 4,963,481 and 5,168,062.

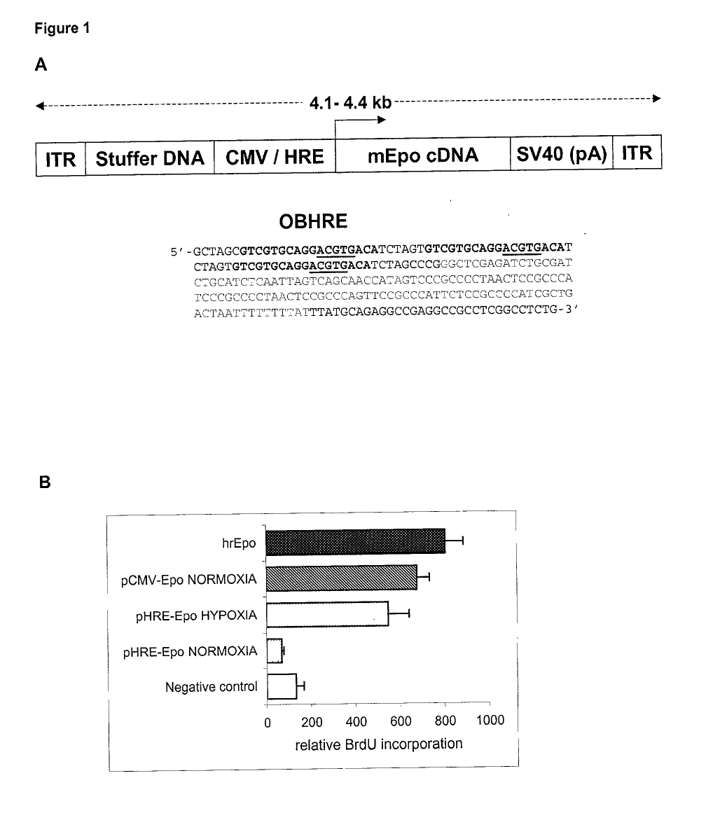

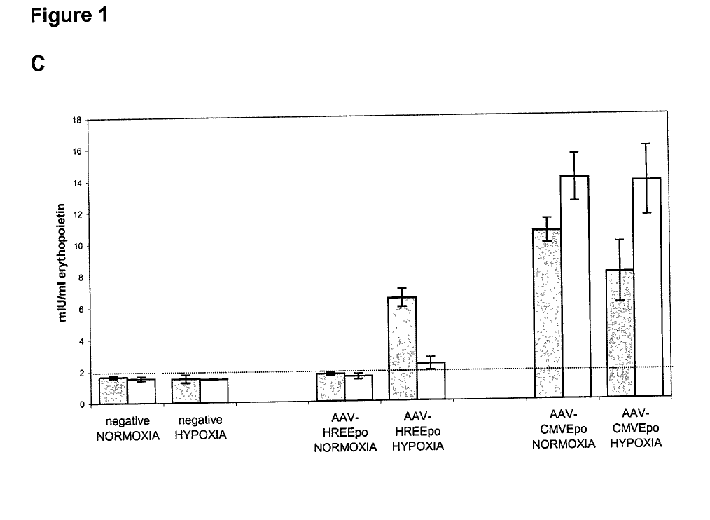

Organization of the construct of the present invention positions an HRE at the 5′ end of the construct optionally in operable linkage with the promoter such that the HRE & promoter (creating a hypoxia inducible promoter/expression control sequence) controls expression of the Epo gene as set forth in FIG. 1A of this specification.

The present invention provides a vector system which can be viral or non viral. Gene delivery of the Epo gene has been accomplished using a variety vectors such as retroviral, lentiviral, adenoviral, adeno-associated viral, naked DNA, lipid-complexed DNA, and biolistic DNA delivery (U.S. Pat. No. 6,211,163, Osada et al. (1999), Dalle et al. (1997), Bohl et al. (1998), EP 1013288, Rudich et al. (May 2000, Zhou et al. (May 1998, Svennson et al. (October 1997), Beall et al. (March 2000), Payen et al. (March 2001, Tripathy et al. (November 1994), Klinman et al. (March 1999), Maione et al. (April 2000), Descamps et al. (August 1994, Maruyama et al. (March 2001), Verma (1999, Kessler et al. (November 1996), Seppen et al. (August 2001, (Bohl et al. (1997), Osborne et al. (August 1995), Villeval et al. (August 1994), Serguera et al. (1999), as above); see also U.S. Pat. Nos. 6,156,567, 6,090,393, 6,004,777, 5,990,091 and 6,130,066, and documents cited in these U.S. Patents, all incorporated herein by reference, for discussions of vectors that can be employed in the practice of the invention, including discussions of canine and human adenoviruses, and other vectors, e.g., poliovirus, herpesvirus, poxvirus, DNA vector, etc. Adenoviruses useful in the practice of the invention can have deletions in the E1 and/or E3 and/or E4 regions, or can otherwise be maximized for receiving heterologous DNA; see, e.g., U.S. Pat. Nos. 6,156,567, 6,090,393, wherein an insertion of heterologous DNA can be in the E3 region or in the region located between the E4 region and the right ITR region. Mention is also made of U.S. Pat. Nos. 6,228,844, 6,214,804, 5,703,055, 5,693,622, 5,589,466, 5,580,859, 5,459,127, 5,264,618, which can involve vectors useful in the practice of the invention.

The present invention further provides for administration of the vector system by any route of administration, such as intramuscular, subcutaneous, intravascular, or intraperitoneal (U.S. Pat. No. 6,211,163, as above, and Seppen (2001), as above).

The present invention provides the use of any Epo coding sequence. This sequence can be synthetic or can be derived from a species of Epo such as human Epo, non-human primate Epo, canine Epo, feline Epo, porcine Epo, bovine Epo, equine Epo, ovine Epo, and murine Epo. It is known that there is a high degree of sequence homology among Epo sequences in mammals. In fact, it has been reported that human Epo is 91% identical to monkey Epo, 85% to cat and dog Epos, and 80% to 82% to pig, sheep, mouse and rat Epos (Wen et al. (1993) Blood 82: 1507-1516). See also, WO99/5486; EP 1013288; U.S. Pat. Nos. 5,952,226; 5,621,080; 5,888,774; 4,954,437; 4,703,008; and 5,547,933; Descamps et al. (1994), as above; Seppen et al. (2001), as above; Shoemaker et al. (1986) Mol. Cell. Biol. 6: 849-858; Beall (2000), as above; Suliman et al. (1996) Gene 171: 275-280; and MacLeod et al. (1998) Am. J. Vet. Res. 59: 1144-1148. Accordingly, the invention is useful for delivery of Epo to humans, and non-human vertebrates, e.g., non-human mammals, such as canines, felines, non-human primates, porcines, bovines, equines, ovines, etc. Indeed, a problem recognized in the art is that human Epo is administered to animals, such as dogs, for treating anemia and/or other maladies, eventually leading to an immune response against the human Epo, such that there is a need for delivery of Epo to a particular species, e.g., species-specific delivery of Epo (such as delivery of canine Epo to dogs); and, the present invention may address this problem by providing to a host a vector that encodes an Epo specific to that host (such as providing to a dog a vector encoding canine Epo), or an Epo in a form that does not give rise to the problems encountered with administering human Epo to animals such as dogs. In such an instance, the vector can be tailored to the host too. For instance, if the intended host is a dog, the vector can be a canine adenovirus, with the coding therein for the Epo advantageously coding for canine Epo.

The present invention also provides modified, truncated, mutein, and active forms of Epo. See, e.g., U.S. Pat. Nos. 5,457,089; 5,166,322; 4,835,260; and 5,106,954. With respect to Epos, see also U.S. Pat. Nos. 5,955,422; 5,756,349; 5621,080; 5,618,698; 5,547,933; 4,703,008; 5,856,298; 5,661,125; 5,106,760; 4,703,008; 5,856,298; 5,661,125; 5,106,760; 4,558,006; 5,574,018; 5,354,934; 5,013,718; and 4,667,016.

The Epo sequence can be, for example, a synthetic RNA/DNA sequence, a codon optimised RNA/DNA sequence, a recombinant RNA/DNA sequence (i.e. prepared by use of recombinant DNA techniques), a cDNA sequence or a partial genomic DNA sequence, including combinations thereof. It need not be an entire coding region. In addition, the RNA/DNA sequence can be in a sense orientation or in an anti-sense orientation. Preferably, it is in a sense orientation. Preferably, the sequence is, comprises, or is transcribed from cDNA. The Epo sequence may encode all or part of the protein of interest (“POI”), or a mutant, homologue or variant thereof. For example, the Epo sequence may encode a fragment which is capable of functioning in vivo in an analogous manner to the wild-type protein.

The term “mutant” includes an Epo amino acid sequence which includes one or more amino acid variations from the wild-type sequence. For example, a mutant may comprise one or more amino acid additions, deletions or substitutions. A mutant may arise naturally, or may be created artificially (for example by site-directed mutagenesis).

Here, the term “homologue” means an entity having a certain homology with the Epo nucleic acid sequence, or which encodes a protein having a degree of homology with the Epo protein. Here, the term “homology” can be equated with “identity”.

In the present context, a homologous sequence is taken to include an amino acid sequence which may be at least 75, 85 or 90% identical, preferably at least 95 or 98% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, an homologous sequence is taken to include a nucleotide sequence which may be at least 75, 85 or 90% identical, preferably at least 95 or 98% identical to the subject sequence. Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalizing unduly the overall homology score. This is achieved by inserting “gaps” in the sequence alignment to try to maximize local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimized alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8 and tatiana@ncbi.nhn.nih.gov).

Although the final % homology can be measured in terms of identity, the alignment process itself is typically not based on an all-or-nothing pair comparison. Instead, a scaled similarity score matrix is generally used that assigns scores to each pair-wise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues which produce a silent change and result in a finctionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | GAP |
| | | ILV |
| | Polar-uncharged | CSTM |
| | | NQ |
| | Polar-charged | DE |
| | | KR |
| AROMATIC | | HFWY |

The present invention also encompasses homologous substitution (substitution and replacement are both used herein to mean the interchange of an existing amino acid residue, with an alternative residue) may occur i.e. like-for-like substitution such as basic for basic, acidic for acidic, polar for polar etc. Non-homologous substitution may also occur i.e. from one class of residue to another.

The present invention provides that the patient to be treated for chronic anemia or age-related anemia may be any patient of a species such as human, non-human primate, canine (e.g., dog, puppy, elder dog), feline (e.g., domestic or household cat, kitten, elder cat), porcine (e.g., pig, boar), bovine (e.g., cow), equine (e.g., horse), ovine (e.g., sheep, lamb), and murine. Advantageously, the present invention finds use in a clinical setting which can include use in the veterinary field providing treatment to companion animals as well as farm and/or production and/or sport animals. Advantageously, the Epo coding sequence and patient to be treated can be of the same species or of different species. While the art recognizes a potential problem in the art based upon the importance of using autologous genes for Epo expression in animal strains with differing immunological responsiveness, the art also recognizes that species differences between host and gene can be tolerated (Kessler et al. (1996) PNAS 93: 14082-14087). To this end, the present invention provides for the physiological regulation of Epo expression in an anemic environment such that it is believed that tight control of Epo expression should overcome the need to limit the method to the use of an Epo gene from the same species in need of such treatment.

More specifically, the present invention has arisen from a desire to seek a model of human clinical potential. To this end, it was reasoned that the Epo-TAg mouse (Maxwell (1993), as above) should have tissue hypoxia as a consequence of the chronic anemic state and that this could be sufficient to activate gene expression from a hypoxia responsive promoter. In theory, once sufficient Epo was produced to restore the red blood cell (RBC) level to normal, the tissues should revert to normoxia and the HRE should cease to drive transcription. This would reduce Epo production and ensure that polycythemia does not develop.

Applicants have now tested this concept by using a recombinant adeno-associated viral (AAV) vector to express murine Epo under the control of a constitutive promoter (CMV) or a hypoxia regulated promoter (HRE). The method of gene delivery was chosen because the vascularity of skeletal muscle allows for the distribution of secreted proteins. In addition, as the hypoxia signalling pathway is functional in muscle, AAV gives a good gene transfer to muscle, and in a clinical setting, skeletal muscle is easily targeted by injection. The effect of intramuscular delivery of these vectors on the hematocrit and organ structure of normal and EpoTAg mice has been assessed over a long term study. The data indicates that Epo can be delivered upon physiological demand to reverse a chronic state of anemia.

The vectors can be administered in quantities based on the Examples herein, or in quantities that are based on the quantities of vector employed in documents cited herein or in other literature or patents, or in quantities for in vivo expression which is commensurate with doses of protein Epo typically given to the particular patient (e.g., human or non-human). The dose for a particular patient can be determined by the skilled artisan, from this disclosure and the knowledge in the art, based on factors typically taken into consideration in the medical and veterinary arts, such as the particular species of the patient, age, sex, weight, condition and nature of host, as well as LD.sub.50 and other screening procedures which are known and do not require undue experimentation. Dosages of expressed product (protein Epo) can range from a few to a few hundred micrograms, e.g., 5 to 500 μg; for instancewhen EPO is administered to a human patient (average mass about 70 kg) subcutaneously, it is given at a dose of about 40,000 units per week and if an inadequate response is seen, the dose can be increased to about 60,000 units, or lowered to about 20,000 units, on a weekly basis, depending on the response generated. The inventive recombinant or vector can be administered in any suitable amount to achieve expression at these dosage levels. The viral recombinants of the invention can be administered in an amount of about 10^{3.5} pfu; thus, the inventive viral recombinant is preferably administered in at least this amount; more preferably about 10⁴ pfu to about 10⁶ pfu; however higher dosages such as about 10⁴ pfu to about 10¹⁰ pfu, e.g., about 10⁵ pfu to about 10⁹ pfu, for instance about 10⁶ pfu to about 10⁸ pfu can be employed. Suitable quantities of inventive plasmid or naked DNA in plasmid or naked DNA compositions can be 1 μg to 100 mg, preferably 0.1 to 10 mg, but lower levels such as 0.1 to 2 mg or preferably 1-10 μg may be employed. The dose can be adjusted or determined so that the patient's hematocrit levels are corrected and/or maintained.

Inventive vectors or formulations containing inventive vectors can be readminstered, e.g., periodically and/or when hematocrit levels of the patient drop below corrected and/or maintained levels.

Inventive vectors may be formulated for administration based on the Examples herein, or based on formulations employed in documents cited herein or in other literature or patents, and can contain excipients, carriers, diluents and the like employed in vector formulations suitable for veterinary or medical (pharmaceutical) purposes, i.e., the formulations can contain veterinarily acceptable and/or pharmaceutically acceptable carrier(s), diluent(s), excipient(s) and the like, such as water or a buffered saline,. physiological saline, glucose or the like with or without a preservative. The vector compositions can also be lyophilized for resuspension or dissolving into solution, e.g., mixture with a carrier, diluent or excipient at or about the time of administration. The compositions can contain auxiliary substances, such as wetting or emulsifying agents, pH buffer agents, gelling or viscosity enhancing additives, preservatives, colors, and the like.

Accordingly, the invention comprehends a kit wherein the vector composition in lyophilized form is provided in a container, and a carrier, excipient or diluent is provided in a separate container, for admixture with the vector, to form a solution or suspension of the vector, for administration. The containers are optionally in the same packaging; and, the kit optionally can include instructions for admixture and/or administration. Thus, the invention further comprehends methods for preparing the vectors, as well as methods for preparing medicaments containing the vectors. The methods for preparing the vectors comprise operably linking the HRE(s) and the Epo coding sequences, optionally with a promoter such as a CMV promoter; and, the methods for preparing the medicaments or formulations comprise admixing the vector with the pharmaceutically and/or veterinarily acceptable carrier, diluent or excipient.

The inventive vector or formulation containing the inventive vector or the Epo expressed from the inventive vector can be administered alone, or in combination with other therapies for anemia or conditions underlying or causing the anemia; and thus, the invention comprehends combination therapy including the inventive vector or a formulation containing an inventive vector or an expression product from an inventive vector.

The invention will now be described by way of the following non-limiting Examples, given by way of illustration.

### EXAMPLES

### Materials & Methods

Normal and Anemic Mice

The generation of the anemic (EpoTAg^{h}) transgenic mice in which the SV40 large T antigen marker gene is integrated in the regulatory sequence of the endogenous mouse Epo gene is described elsewhere (Maxwell (1993, as above). The breeding colony of Epo-TAg^{h} and normal (C57B16/CBA) mice used in this study was maintained at CAMR, Porton Down, Wiltshire. The female EpoTAg^{h} homozygote mice were generated from F1 breeding pairs of heterozygote females and homozygote males. The genotype was determined by hematocrit; homozygote 17.5 +/−4%, heterozygote 35.5 +/−4.1% compared to the normal 52%.

Cell Lines

The T47D and HT1080 cell lines (ECACC, Wiltshire, UK) were used to assess hypoxic regulation of the Epo expression vectors since they have previously been shown to show good hypoxic induction in vitro 22. The cells were maintained in RPMI 1640 or Dulbecco's modified Eagle's medium respectively supplemented with 10% (v/v) fetal calf serum, 2 mM glutamine and 2 mM non-essential amino acids (Sigma-Aldrich, Dorset, UK).

Transient Transfections

Typically, cells seeded in a 24-well dish were brought to 70% confluence and transfected with 0.21 μg of plasmid using the Fugene-6 transfection reagent (Boehringer Mannheim, Indianapolis, USA).

Hypoxia in vitro

24 hours post-transduction or transfection, cells were either incubated for a further 16 hours under normoxic conditions in a standard incubator (21% O₂, 5%CO₂, 74% N₂) or under hypoxic conditions (0.1% O₂, 5% CO₂, 95% N₂) using a multigas incubator purchased from Heto-Holten (Allerod, Denmark).

In Vitro Biological Assay for Erythropoietin

The functionality and regulation of the cloned Epo cDNA was verified using a biological spleen cell proliferation assay based on a published method (Krystal (1983) Exp. Hematol. 11: 649-660). Briefly, 2 to 3 month old mice (C57BL/6J×C3H/HeB) Fl hybrid weighing 25-35 g were given two consecutive daily intraperitoneal injections of 60 mg/kg phenylhydrazine hydrochloride. Spleens were isolated three days after the second injection. Single cell suspensions from the spleen were prepared 3 days after the second injection and seeded into black-walled 96-well plates black plates (Canberra Packard, Ontario, Canada) at a density of 4×10⁵ cells per well. Supernatants were collected from HT1080 cells five days post-transfection with either pCMV-EPO or pHRE-EPO plasmids and 1 μl added to the splenocyte cell cultures. As a positive control recombinant human Epo (rhEpo) was used at 500 U/ml. The splenocyte cell cultures were incubated for 22 hrs and then assayed for proliferation using a chemiluminescent BrdU assay (Roche, Mannheim, Germany).

Detection of Erythropoietin In Vitro

Erythropoietin was detected in cell supernatants using the Quantikine IVD Epo Elisa kit, detectable threshold 2 mU/ml, (R & D systems, Abingdon, Oxon).

Histological Analyses

Standard haematoxylin and eosin staining was carried out in order to assess cell morphology. For immunohistological analysis the tissue sections were air dried and then fixed in absolute ethanol for 10 minutes. Endogenous peroxidase activity was blocked with 0.3% H₂O₂ in methanol for 10 minutes. To block non-specific binding sections were incubated in normal goat serum for 10 minutes followed by incubation with the primary antibody. Rabbit polyclonal VEGF (Santa-Cruz, Sc-507) was used at a dilution of 1/10. Goat anti rabbit horseradish peroxidase conjugated secondary antibody was used at a dilution of 1/50. Peroxidase substrate (DAB, Vector) was added for 10 minutes, washed and then counterstained using Gill's haematoxylin. Biotinylated mouse monoclonal CD 31(BD Biosciences, 09332A) was used at a dilution of 1/100. Staining was detected using an alkaline phosphatase conjugated streptavidin secondary antibody at a dilution of 1/300. Slides were washed in distilled water for 5 minutes and then incubated in NBT/BCIP substrate (Roche). Levamisole was added to this solution to block endogenous alkaline phosphatase activity as per the manufacturer's instructions. Slides were counterstained in Gill's haematoxylin.

The percentage of CD31 positive cells in the tissue sections was calculated by random, equally processed digital images using the Aequitas Image Analysis Software (Digital Data Ltd., Cambridge, UK).

For electron microscopy the hearts were dissected in to 1 mm cubes and immersion fixed in 1% gluteraldehyde/2.5% paraformaldehyde. Samples were washed in PBS and post fixed in 1% OsO4 in 0.1M phosphate buffer for 40 minutes, washed in distilled water overnight at 4 C., dehydrated in alcohols and embedded in Durcupan resin. Ultra thin cross-sections of the myocardium were stained with uranyl acetate, followed by 1% lead citrate (Reynold's stain), and examined under the Philips 401 transmission electron microscope.

### Example 1

### Construction of Recombinant AAV Vectors

The murine erythropoietin cDNA was cloned via nested PCR on murine kidney cDNA (Quickclone cDNA, Clontech, UK) using two pairs of nested PCR primers:

| | |
|---|---|
| Primer set 1: | |
| 5′-GACAGTGACCACTTTCTTCCAG-3′, | (SEQ ID NO: 1) |
| 5′GGACAGACTGGTAAGAAGGTAATG-3′. | (SEQ ID NO: 2) |
| Primer set 2: | |
| 5′-CAGCTAGGCGCGGAGATG-3′, | (SEQ ID NO: 3) |
| 5′-CAGCAGCATGTCACCTGTC-3′. | (SEQ ID NO: 4) |

The mEpo PCR product was cloned in to the pUC18 plasmid (Panvera Corp, Wisconsin, USA) and was subsequently removed as an XbaI-EcoRI fragment and cloned into the pCI-Neo (Promega, Southampton, UK) NheI-EcoRI sites to create pCMV-Epo. The CMV/IE promoter in pCMV-Epo was replaced with the OBHRE promoter (Boast et al. (1999) Hum. Gene Ther. 10: 2197-2208) to create pHRE-Epo. An oligonucleotide was cloned into the BamHI and SpeI restriction sites in the multiple cloning site of the

pSL1180 plasmid (Amersham Pharmacia Biotech, Buckinghamshire, UK) to generate the following restriction sites: BamHI-NheI-MluI-XhoI-StuI-NruI-BclI-SpeI-BglII.

The AAV-CMVEpo vector genome was constructed by creating a145 bp oligonucleotide consisting of the wild-type AAV-2 inverted terminal repeat (ITR) (Genbank Accession number: NC_{—}001401) flanked by BamHI and NheI compatible ends. The ITR was cloned sequentially in both reverse and forward orientation into the BamHI-NheI and SpeI and BglII sites of the modified pSL1180 vector. The CMV-Epo BsaBI-BglII fragment from pCMVEpo was cloned into the StuI-BglII sites of the modified pSL1180 vector together with a 1.7 kb BclI-BglI stuffer fragment from the LacZ gene such that the complete internal cassette is 4.2 kb. The AAV-HREEpo vector genome was created by exchanging the CMV/IE NotI-Eco47III promoter fragment in AAV-CMVEpo for the OBHRE NotI-XmnI promoter fragment in pHRE-Epo (FIG. 1A).

The recombinant AAV-2 vectors were produced according to the published method (Zhang et al. (1999) Hum. Gene Ther. 10: 2527-2537). AAV particles were determined by dot blot quantification of genome copy and direct comparison to a recombinant AAV vector expressing CMV-GFP of known biological titer.

### Example 2

### Hypoxia Mediated Regulation of Functional Murine Epo Expression In Vitro

It was observed that a synthetic HRE multimer referred to as OBHRE can combine a good induction ratio with high level of expression comparable to that achieved by strong constitutive promoters such as the CMV promoter but only when the oxygen concentration is low (Boast et al. (1999), as above). The OBHRE promoter was inserted into plasmid and AAV-2 vectors to produce pHRE and AAV-HRE respectively (FIG. 1a). Similar vectors containing the human CMV promoter are pCMV and AAV-CMV. A cDNA for murine Epo was inserted into these vectors and GFP expressing vectors were used as negative controls. Murine Epo rather than human Epo was used to ensure that immune responses would not compromise the efficacy of the gene therapy. It was first confirmed that the murine Epo gene functioned in vitro. The production of mEpo in the culture supernatant of HT1080 cells, transfected with pHRE-Epo or pCMV-Epo and maintained in normoxia or hypoxia, was determined using a spleen cell proliferation assay (FIG. 1b). Both plasmids directed the expression of functional mEpo, but in the case of the pHRE-Epo, the expression was eight fold higher from cells maintained in hypoxia as compared to the cells maintained in normoxia. Similarly, the recombinant AAV vectors were transduced into T47D cells, placed in normoxia or exposed to hypoxia for 16 hours and then returned to normoxia (FIG. 1c). The secretion of mEpo into the supernatant was assessed in an Epo ELISA 1 day and 4 days after hypoxic induction. AAV-CMV directed mEpo expression increased during the four days in both normoxia and hypoxia whereas AAV-HRE directed mEpo expression increased from basal levels up to a similar maximum level only in the hypoxia exposed cultures as measured at day 1. By day 4, however, levels of mEpo had returned to baseline. These data indicated that by two assays the mEpo gene was functional and that the expression could be activated by hypoxia and switched off in normoxia. This reversible expression was the profile that would be required for a gene therapy vector that could deliver Epo under anemic conditions, but which would be shut down once normal oxygenation was restored.

### Example 3

### Hypoxia Mediated Regulation of Functional Murine Epo Expression In Vivo

Hypoxic Status of Skeletal Muscle in Epo-TAg Mice

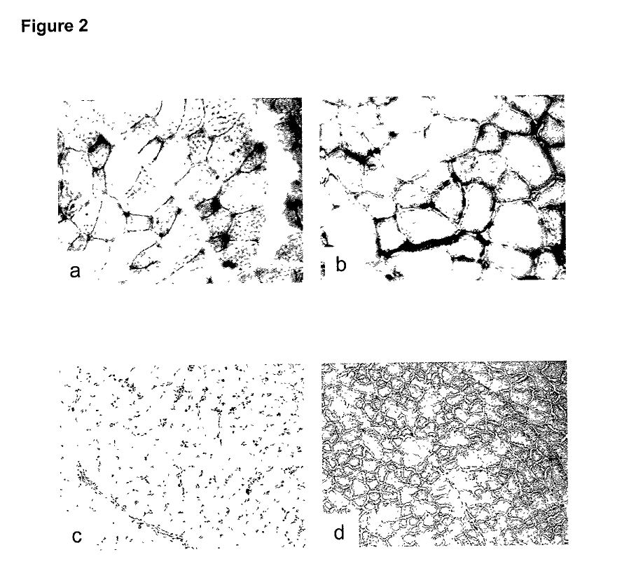

The concept of using a hypoxia responsive promoter to drive mEpo expression in skeletal muscle requires that there is tissue hypoxia. This was assessed prior to the study by examining the muscle for the expression of vascular endothelial growth factor (VEGF) and the consequent hypervascularisation. VEGF gene expression is activated by hypoxia, predominantly via the HIF-1 mediated transcriptional pathway, and stimulates endothelial cell proliferation and neovascularisation. This presumably is an attempt to compensate for the low oxygen tension in the tissue by increasing the blood flow/oxygen supply to the anemic limb. Hind limb skeletal muscle from the EpoTAg^{h} mice showed increased staining foT VEGF and for CD31, an endothelial cell specific marker from 10.7% +/−5.1 in the EpoTAg^{h} compared to 7.4% +/−4.0 in the normal skeletal muscle (FIG. 2). These data indicated that the skeletal muscle was overexpressing VEGF and was therefore likely to be sufficiently hypoxic to activate the HRE, particularly in the young mice at the start of the study.

Regulated Delivery of Epo In Vivo

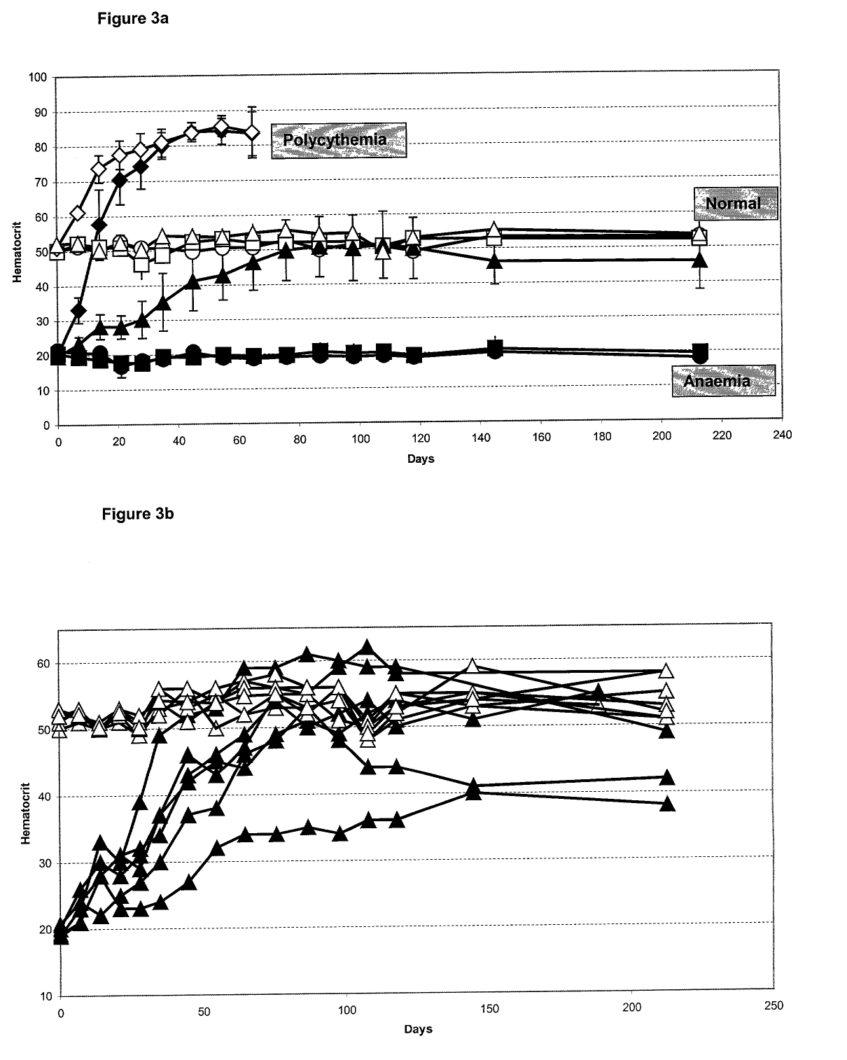

Twelve week old female mice were injected with a total dose of 1×10¹⁰ particles of recombinant AAV vector at four sites in the left hind-limb. Two 30 μl injections were made in to the quadriceps and two 20 μl in to the anterior tibialis muscles. Hematocrit measurements were made regularly over a period of 7 months (FIG. 3). The control vector was AAV-CMVGFP and this produced no change in the hematocrit in normal mice, which was maintained at about 52% (FIG. 3a, open squares) or EpoTAg^{h} mice which was maintained at about 18% (FIG. 3a, closed squares) throughout the duration of the study. These levels were identical to the untreated controls (FIG. 3a, normal mice, open circles; EpoTAg^{h} mice, closed circles). In marked contrast, when the normal and EpoTAg^{h} mice were injected with the constitutive Epo vector, AAV-CMVEpo, there was a dramatic rise in the hematocrit in both groups that was significant at 5 days and that increased to 85% after 35 days (FIG. 3a, diamond symbols). Two mice in this group died suddenly at day 60, by which time the blood in the remaining animals became too viscous to obtain samples for hematocrit analysis so the animals in these groups were sacrificed. However, a dramatically different result was obtained when the hypoxia regulated vector, AAV-HREEpo, was used. In normal mice (FIG. 3, open triangles) there was no effect on the hematocrit, it was virtually indistinguishable from the untreated and AAV-CMVGFP treated controls giving a peak hematocrit of 55.6% +/−1.8 at day 78. In the EpoTAg^{h} mice, the hematocrit began to rise steadily until at 75 days a plateau was reached. This plateau was at an average hematocrit of 54%, i.e., in the normal range (FIG. 3, closed triangles). This normal hematocrit was maintained up to 160 days when the study terminated. The response was remarkably consistent across all the treated animals and the individual data are shown in FIG. 3b. The hematocrits of the AAV-HREEpo treated normal mice are virtually super impossible. The hematocrits of the AAV-HREEpo treated EpoTAg^{h} mice showed some variation in terms of the rate of increase and plateau level. However, in no case did the hematocrit reach the levels obtained by the constitutive vector, and in all cases, plateau levels were within the normal range. The constitutive AAV-CMVEpo vector was highly toxic causing death or severe morbidity by 65 days. Whereas treatment with the hypoxia regulated AAV-HREEpo vector not only restored normal hematocrit, but also lead to the maintenance of these normal levels for the duration of the 7 month study.

Organ Analysis of Animals

It was desirable to determine if Epo gene therapy caused any structural changes to internal organs. Changes in red blood cell composition affect both the volume and pressure of the blood. In chronic anemia, this hemodynamic alteration leads to gradual development of cardiac enlargement (hypertrophy) as the cardiac output increases to compensate for the decreased oxygen carrying capacity of the blood. A significant increase in the hematocrit, a condition known as polycythemia greatly increases the viscosity of the blood leading to greater risk of thrombosis and heart failure.

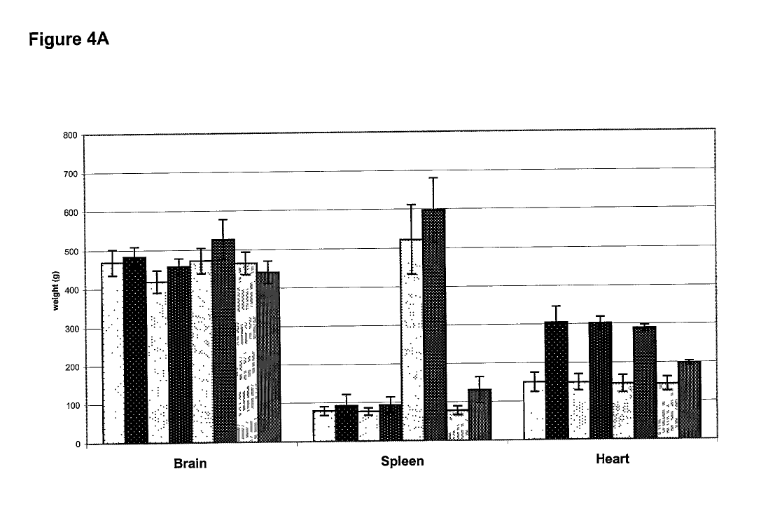

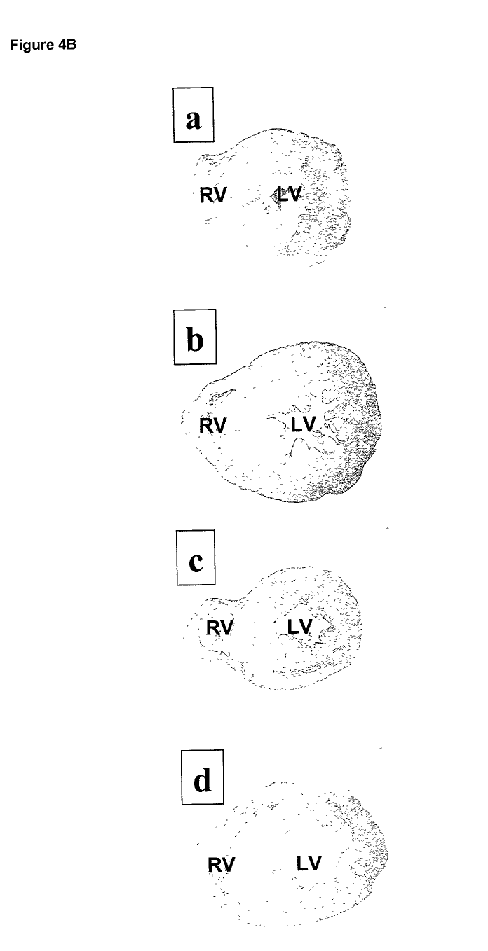

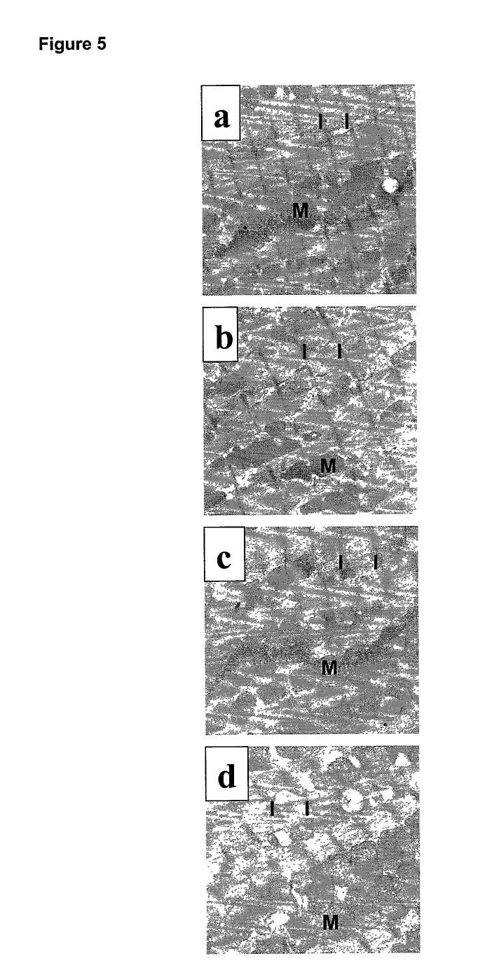

The weights of some of the organs in the untreated and treated EpoTAg^{h} and normal mice (FIG. 4a) were compared. There was no difference between any of the groups in the size of the brains. However, marked differences were noted in the spleen. The EpoTAg^{h} mice had spleens that were 70% smaller than the normal mice consistent with the reduction in circulating RBCs. The AAV-CMVEpo treated normal and EpoTAg^{h} mice had massively enlarged spleens (splenomegaly), most likely as a result of vascular congestion due to the increase in RBC load. Splenomegaly has a high incidence (70%) in patients suffering from polycythemia.

There was a doubling of the heart size in the EpoTAg^{h} mice compared to normal consistent with anemia associated hypertrophy. Over-production of Epo from the AAV-CMVEpo vectors caused a further 30% increase in the heart weight of the EpoTAg mice and caused the hearts of the normal mice to increase by 56%. This is presumably due to vascular congestion causing edema in these organs.

Ultrastructure analysis of the hearts confirmed gross hypertrophy in the EpoTAg^{h} mice. Hypertrophy is an increase in the size of a tissue due to increased size of individual cells. It occurs in tissues made up of permanent cells, in which a demand for increased metabolic activity cannot be met through cell multiplication.

In summary, this study describes the surprising and unexpected results obtained by the physiologically-regulated expression of Epo by an HRE. In particular, this study supports that gene therapy by delivery of a recombinant HREEpo vector provides long-term physiologically-regulated expression of Epo for correction of the hematocrit in a genetically anemic environment, without the requirement for any other external intervention or management.

Having thus described in detail preferred embodiments of the present invention, it is to be understood that the invention defined by the appended claims is not to be limited by particular details set forth in the above description as many apparent variations thereof are possible without departing from the spirit and scope thereof.

Reference List

Maxwell et al. (1993) Kidney Int. 44: 1149-1462

Ebert et al. (1999) Blood 94: 1864-1877

Wenger et al. (1997) Biol. Chem. 378: 609-616

Binley et al. (1999) Gene Ther. 6: 1721-1727

Griffiths et al. (2000) Gene Ther. 7: 255-262

Shibata et al. (2000) Gene Ther. 7: 493-498

Goodnough et al. (2000) Blood 96: 823-833

Bron et al. (2001) Semin. Oncol. 28: 1-6

Osada et al. (1999) Kidney International 55: 1234-1240

Dalle et al. (1997) Hematol. Cell Ther. 39: 109-113

Bohl et al. (1998) Blood 92: 1512-1517

Rudich et al. (May 2000) J. Surg. Res. 90: 102-108

Zhou et al. (May 1998) Gene Ther. 5: 665-670

Svennson et al. (October 1997) Hum. Gene Ther. 8: 1797-1806

Beall et al. (March 2000) Gene Ther. 7: 534-539

Payen et al. (March 2001) Exp. Hematol. 29: 295-300

Tripathy et al. (November 1994) PNAS 91: 11557-11561

Klinman et al. (March 1999) Hum. Gene Ther. 10: 659-665

Maione et al. (April 2000) Hum. Gene Ther. 11: 859-868

Descamps et al. (August 1994) Hum. Gene Ther. 5: 979-985

Maruyama et al. (March 2001) Gene Ther. 8: 461-468

Verma (1999) J. Gene Med. 1: 64-66

Kessler et al. (November 1996) PNAS 93: 14082-14087

Seppen et al. (August 2001) Blood 98: 594-596

Bohl et al. (1997) Nat. Med. 3: 299-305

Osborne et al. (August 1995) PNAS 92: 8055-8058

Villeval et al. (August 1994) Blood 84: 928-933

Serguera et al. (1999) Hum. Gene Ther. 10: 375-383

Bohl et al. (2000) Blood 95: 2793-2798

Dalle et al. (1999) Gene Ther. 6: 157-161

Hamamori et al. (1995) J. Clin. Invest. 95: 1808-1813

Ye et al. (1999) Science 283: 88-91

Setoguchi et al. (1994) Blood, 94: 2946-2953

Aebischer et al. (1997) Human Gene Therapy, 8(16): 1840-1841

Semenza et al. (1992) PNAS 88: 5680-5684

Blanchard et al. (1992) Mol. Cell. Biol. 12: 5373-5385

Wen et al. (1993) Blood 82: 1507-1516)

Shoemaker et al. (1986) Mol. Cell. Biol. 6: 849-858

Suliman et al. (1996) Gene 171: 275-280

MacLeod et al. (1998) Am. J. Vet. Res. 59: 1144-1148

Kessler et al. (1996) PNAS 93: 14082-14087

Krystal (1983) Exp. Hematol. 11: 649-660

Zhang et al. (1999) Hum. Gene Ther. 10: 2527-2537

Boast et al. (1999) Hum. Gene Ther. 10: 2197-2208

Kauftnan et al. (1998) N Engl J Med 339: 578-583

Anemia Work Group. (1997) Am J Kidney Dis. 30(4 suppl 3): S192-S240

Asha et al. (1993) Am J Kid Dis. 22(2 suppl 1): 23-31

Raine et al. (1991) Am J Kidney Dis. 18(4 suppl 1): S76-S83

Besarab et al. (1998) N Engl J Med. 339: 584-590

Watson et al. (1990) Am J Med. 89: 432-435

1. A method for treating anemia in a patient in need thereof, the method comprising, administering to said patient, a vector comprising a nucleic acid sequence encoding erythropoietin (Epo) in operable linkage with an HRE expression control sequence, wherein expression of Epo is physiologically regulated such that hematocrit levels of said patient are corrected and maintained.

2. The method of claim 1, wherein the vector is a viral vector.

3. The method of claim 2, wherein the viral vector is an adeno-associated viral vector.

4. The method of claim 1, wherein the HRE expression control sequence is an Epo HRE expression control sequence.

5. The method of claim 1, wherein the HRE expression control sequence is a PGK-1 HRE expression control sequence.

6. The method of claim 1, wherein the HRE expression control sequence is a LDH-A HRE expression control sequence.

7. The method of claim 1, wherein the HRE expression control sequence is in operable linkage with a promoter.

8. The method of claim 7, wherein the promoter is a viral promoter.

9. The method of claim 8, wherein the viral promoter is the CMV promoter.

10. The method of claim 1, wherein the HRE expression control sequence includes two or more HRE expression control sequences.

11. The method of claim 10 wherein the HRE expression control sequence is a PGK-1 HRE expression control sequence.

12. The method of claim 1 wherein the patient is a human.

13. The method of claim 1 wherein the patient is a non-human mammal.

14. The method of claim 13 wherein the patient is a canine, feline, bovine, equine, ovine, porcine or non-human primate.

15. A vector system comprising a nucleic acid sequence encoding erythropoietin (Epo) in operable linkage with an HRE expression control sequence, wherein the HRE expression control sequence includes two or more HRE expression control sequences; and, the vector system, when administered to a host, provides for physiological regulation of Epo.

16. The vector system of claim 15, wherein the vector is a viral vector.

17. The vector system of claim 16, wherein the viral vector is an adeno-associated viral vector.

18. The vector system of claim 15, wherein the HRE expression control sequence is an Epo HRE expression control sequence.

19. The vector system of claim 15, wherein the HRE expression control sequence is a PGK-1 HRE expression control sequence.

20. The vector system of claim 15, wherein the HRE expression control sequence is a LDH-A HRE expression control sequence.

21. The vector system of claim 15, wherein the HRE expression control sequence is in operable linkage with a promoter.

22. The vector system of claim 21, wherein the promoter is a viral promoter.

23. The vector system of claim 22, wherein the viral promoter is the CMV promoter.
